(19) United States
(12) Patent Application Publication
(10) Pub. No.: US 2026/0199553 A1
(43) Pub. Date: Jul. 16, 2026

(54) 3D FAT TISSUE STRUCTURE, PRODUCED USING LIGHT-BASED STRUCTURING CURING, FOR USE AS A BREAST IMPLANT

(71) Applicant: Cellbricks GmbH, Berlin (DE)

(72) Inventors: Anna-Klara Amler, Berlin (DE); Melissa Freitag, Berlin (DE); Marius Friedel, Berlin (DE); Lutz Kloke, Berlin (DE); Tobias Lam, Berlin (DE); Alexander Thomas, Berlin (DE); Nina Hedemann, Kiel (DE); Marion Tina van Mackelenbergh, Dänischenhagen (DE)

(21) Appl. No.: 19/134,529

(22) PCT Filed: Dec. 6, 2023
(86) PCT No.: PCT/DE2023/100942

§ 371 Date: May 30, 2025

(30) Foreign Application Priority Data
Dec. 7, 2022 (DE) 10 2022 132 519.9

(51) Int. Cl. A61L 27/36; B33Y 80/00

(52) U.S. Cl. CPC A61L 27/3604; A61L 27/3645; A61L 27/3687; B33Y 80/00; A61L 2430/04

(57) ABSTRACT

The invention relates to a 3D adipose tissue scaffold for use as a breast implant for surgical, therapeutic, prophylactic or aesthetic restoration, reconstruction or replacement of human breast tissue, wherein the 3D adipose tissue scaffold comprises a matrix of a biocompatible polymer which is colonized with a plurality of biological cell types, the colonization with the plurality of cell types taking place during the construction of the matrix of biocompatible polymer with the formation of a blood vessel network within the 3D adipose tissue scaffold, characterized in that light-based, structuring curing of a photopolymerizable or photocrosslinkable liquid is used for the construction of the matrix of biocompatible polymer. The invention also relates to the use of a 3D adipose tissue scaffold as a breast implant in humans, wherein the 3D adipose tissue scaffold comprises a matrix of biocompatible polymer which is colonized with a plurality of cell types, wherein the colonization with the plurality of cell types takes place during the construction of the matrix of biocompatible polymer with the formation of a blood vessel structure network within the 3D adipose tissue scaffold, characterized in that light-based, structuring curing of a photopolymerizable or photocrosslinkable liquid is used for the construction of the matrix of biocompatible polymer.

### REFERENCE TO PENDING PRIOR PATENT APPLICATIONS

This patent application is a 371 national stage entry of pending prior International (PCT) Patent Application No. PCT/DE2023/100942, filed 6 Dec. 2023 by Cellbricks GmbH for 3D FAT TISSUE STRUCTURE, PRODUCED USING LIGHT-BASED STRUCTURING CURING, FOR USE AS A BREAST IMPLANT, which patent application, in turn, claims benefit of German Patent Application No. 10 2022 132 519.9, filed 7 Dec. 2022. The two (2) above-identified patent applications are hereby incorporated by reference.

### FIELD OF THE INVENTION

The present invention relates to a 3D adipose tissue scaffold for use as a breast implant for surgical, therapeutic, prophylactic or aesthetic restoration, reconstruction or replacement of human breast tissue, which is produced by light-based, structuring curing of a photopolymerizable or photocrosslinkable liquid to form a matrix of biocompatible polymer. Furthermore, the present invention also relates to the use of a 3D adipose tissue scaffold as a breast implant (e.g. in the field of aesthetic breast modification/enlargement), which is produced by light-based, structuring curing of a photopolymerizable or photocrosslinkable liquid into a matrix of biocompatible polymer.

### BACKGROUND OF THE INVENTION

With around 2.3 million cases worldwide in 2020, breast cancer is the most commonly diagnosed type of cancer and affects one in eight women in their lifetime. Despite this, mortality rates in Germany have been falling steadily in the 40-70 age group since the late 1990s and the 10-year survival rate after initial diagnosis was over 80% in 2018. One of the standard therapies is the complete surgical removal of the mammary gland (mastectomy), although in many cases the skin and nipple can be preserved, allowing for more natural forms of breast reconstruction. Conventional heterologous breast reconstruction relies on saline or silicone-filled implants, but these are not a lifelong solution and carry high risks of complications such as infection, capsular fibrosis, dislocation, pain and functional limitations in the shoulder/arm area. As an alternative to implants, it is possible to reconstruct the removed breast using an autologous flap graft, whereby tissue from another part of the body is transferred to the chest wall and adapted there. However, this procedure is considerably more complex and time-consuming, requires specific training for the surgeon and involves risks at the donor site such as the risk of muscle weakness, the formation of hernias and conspicuous scars in exposed areas of the body, as well as risks to the transplant such as the death of the body's own tissue.

To overcome the limitations of conventional reconstruction methods, the ideal solution is ideally a complete, biological reconstruction with a living implant.

Of the many techniques used in tissue engineering (tissue construction), the combination of 3D printing technology with biological materials and cells, known as bioprinting, is the most promising method for generating such complex tissue composites. The patient's own (autologous) tissue should be obtained from the patient and used in combination with the latest biofabrication technology to reconstruct the breast without causing the usual discomfort at the donor site during flap surgery. Alternatively, allogeneic material can also be used.

In comparison to other organs, restoration of the original function of the glandular tissue can be neglected in breast reconstruction if necessary—the decisive factors for successful treatment are quality of life, restoration of integrity, aesthetics and haptics. The focus of current research is therefore the generation of true-to-scale, vital, shape- and volume-stable adipose tissue that does not cause fibrotic capsule formation and can be assimilated by the body.

A further development of classic saline or silicone-filled breast implants, which have no biological activity, are hybrid approaches in which synthetic and biological material is combined in the implant. For example, there are numerous studies in which a shaping and shape-retaining 3D support framework made of biodegradable polymers, such as polycaprolactone, is augmented with the patient's liposuctate after implantation. The disadvantage of this method is the support material used, which is biocompatible but takes up to three years to be completely degraded by the body. It is also significantly stiffer than natural adipose tissue and is therefore perceived as a foreign body. As a foreign material, perioperative complications such as inflammation and seroma are also to be expected.

Due to the disadvantages of hybrid breast adipose tissue reconstruction, recent research in this field is pursuing approaches that rely on alternative materials. Hydrogels have the greatest potential, as they come very close to the extracellular matrix of tissues. Living cells can be placed directly on these matrices and behave in a similar way to native tissues. In order to reproduce the complexity of anatomical structures, it is necessary to precisely control the spatial arrangement of cells. Modern 3D printing processes enable this indirectly by controlled deposition of biomaterial along a previously designed construct architecture (production of a hydrogel scaffold) followed by colonization with living cells. This has so far been achieved, for example, by using recombinant type I collagen from genetically modified tobacco plants in extrusion-based 3D printers. The resulting 3D-printed hydrogel scaffolds made of collagen and synthetic polymers are then colonized with cells. However, due to the excellent biocompatibility of hydrogels, it would be desirable to print living cellular material directly into a desired shape, which would make a downstream colonization step obsolete.

However, this approach has not yet been successfully implemented. The direct combination of autologous fat with biomaterials is intended to ensure efficient healing of the bioimplant by the body and the formation of the body's own tissue. This would be associated with a rapid and permanent improvement in quality of life after mastectomy, which would ideally simplify future breast examinations and make reconstructive follow-up operations obsolete.

Regenerative medicine is concerned with curing various diseases by restoring dysfunctional cells, tissues and organs. Tissue engineering focuses on biological replacement, for example with the help of cultivated tissue. In recent years, the fields of application of regenerative medicine have increasingly expanded to include the replacement of tissue, whereas originally this area was mainly made up of stem cell therapies, e.g. for the treatment of leukemia.

The structure of these three-dimensional tissues and cell networks is becoming increasingly complex. Cellular self-organization can no longer guarantee the formation of these complex structures. Therefore, according to the invention, technical aids such as 3D printing, in this case more precisely “bioprinting”, are used. Bioprinting allows the positioning, shaping and production of complex constructs by 3D printing with cells and biopolymers. However, due to the complexity of these processes, the state of the art has not succeeded in producing a corresponding tissue that can be successfully produced in vitro for subsequent implantation into the human body.

### SUMMARY OF THE INVENTION

It is therefore the object of the present invention to provide a 3D adipose tissue scaffold (3D scaffold) for use as a breast implant which overcomes the aforementioned disadvantages. In particular, it is the task of the present invention to provide a 3D adipose tissue scaffold that closely resembles natural breast adipose tissue. Here, according to the invention, it is desirable for the 3D scaffold to have a tissue structure, both geometrically and in terms of material, in which dedicated compartments of, on the one hand, adipose tissue and, on the other hand, vascular structural tissue with a continuous blood vessel network are present.

According to the invention, a 3D adipose tissue scaffold for use as a breast implant for surgical, therapeutic or prophylactic restoration, augmentation or replacement of human breast tissue is provided for this purpose, wherein the 3D adipose tissue scaffold comprises a matrix of a biocompatible polymer which is colonized with a plurality of biological cell types, wherein the colonization with the plurality of cell types takes place during the construction of the matrix of biocompatible polymer with the formation of a blood vessel network within the 3D adipose tissue scaffold, characterized in that light-based, structuring curing of a photopolymerizable or photocrosslinkable liquid is used for the construction of the matrix of biocompatible polymer.

In other words, the present invention also relates to the use of a 3D adipose tissue scaffold for the manufacture of a breast implant for the surgical or therapeutic reconstruction, augmentation or replacement of human breast tissue, wherein the 3D adipose tissue scaffold comprises a matrix of a biocompatible polymer which is colonized with a plurality of biological cell types, wherein the colonization with the plurality of cell types takes place during the construction of the matrix of biocompatible polymer with the formation of a blood vessel structure network within the 3D adipose tissue scaffold, characterized in that light-based, structuring curing of a photopolymerizable or photocrosslinkable liquid is used for the construction of the matrix of biocompatible polymer.

Surgical or therapeutic restoration or replacement shall be understood herein to mean that the abnormal breast tissue removed from a patient is restored or replaced by inserting a breast implant. The (surgical or therapeutic or prophylactic) augmentation of human breast tissue is also to be understood herein as breast augmentation, i.e. that a breast implant is inserted in addition to the existing breast tissue. The latter can be performed for medical reasons, but also for aesthetic reasons.

In one embodiment of the 3D adipose tissue scaffold according to the invention, it is preferred that the human breast tissue has been removed as a result of a disease. This can involve the partial removal of breast adipose tissue, but also the complete removal of the breast adipose tissue including the mammary gland. It is particularly preferred that the disease is a tumor disease, especially breast cancer.

The term “made of biocompatible polymer” is preferably understood to mean that the 3D scaffold is built up by a matrix of one or more biocompatible polymers.

By “a plurality of cell types” it should be understood that the 3D scaffold according to the invention comprises different cell types that mimic different properties of healthy adipose tissue in a manner as similar as possible to the body.

By “colonization with the plurality of cell types during the build-up of the matrix of biocompatible polymer” is to be understood herein that the various cell types are not introduced only after the build-up of the matrix, i.e. the matrix is colonized, but the colonization with the various cell types takes place simultaneously with the light-based, structuring curing of the photopolymerizable or photocrosslinkable liquid to the matrix of biocompatible polymer.

The light-based, structuring curing of a photopolymerizable or photocrosslinkable liquid to form the biocompatible polymer can be any exposure process that is suitable for building up a matrix of a biocompatible polymer. In this process, a photopolymerizable or photocrosslinkable liquid is cured by the structuring irradiation of light (electromagnetic radiation) to form a biocompatible polymer. In particular, according to the invention, a stereolithographic 3D printing process is used for the light-based, structuring curing, especially preferably one which is a projection-based exposure process, i.e. in which the curing takes place in a focal plane, i.e. preferably it is non-point curing. Such a method is described in EP 3 018 531 A1 and is preferably used according to the invention.

For this purpose, it is preferred that the one or more photopolymerizable or photocrosslinkable liquids comprise at least one cell type and one photopolymerizable or photocrosslinkable substance.

In one embodiment of the 3D scaffold according to the invention, it is therefore preferred that its structure is produced by a stereolithographic 3D printing process. A stereolithographic 3D printing process is understood to be one in which the structure of the 3D scaffold is gradually produced by point-by-point, layer-by-layer (in one layer) or holographic curing, with curing within one layer being preferred according to the invention.

In one embodiment of the 3D scaffold according to the invention, it is preferred that the matrix of the biocompatible polymer is built up in the following step:
(i) Curing of a photopolymerizable or photocrosslinkable liquid by irradiation of an electromagnetic radiation selectively, in a layer or holographically, in the region of the photopolymerizable or photocrosslinkable liquid in which the curing or the build-up of the 3D-scaffold is desired, the irradiation in a layer being preferred.

Irradiation in a layer is understood herein to mean in particular that the 3D scaffold to be built according to the invention is irradiated over its entire surface while it is in the photopolymerizable or photocrosslinkable liquid. Only the areas of the surface where curing is desired are irradiated. Areas of the surface in which no curing is desired can also not be irradiated. In a further step (i), however, an area with a different structure than in the first step (i) can also be produced in the same layer of the 3D scaffold to be built up according to the invention by introducing the layer into a further photopolymerizable or photocrosslinkable liquid and irradiating the desired area of the layer accordingly. Furthermore, the 3D scaffold according to the invention can also be built up by repeating step (i) and applying further cured areas of the same or different structure and/or composition to the already cured areas. In this way, a complex 3D framework with different structures in each layer can be obtained.

Digital light processing is the preferred method for curing in one layer (projection-based or flat).

Preferably, the photopolymerizable or photocrosslinkable substance is present in the photopolymerizable or photocrosslinkable liquid in liquid form, for example dissolved in a solvent.

So that the matrix of the biocompatible polymer can be populated with a plurality of cells, the photopolymerizable or photocrosslinkable liquid used in each case has one or more cell types.

Step (i) is preferably repeated by renewed irradiation of electromagnetic radiation at a point, in a layer or holographically, with irradiation in a layer being preferred according to the invention. At the repetition of step (i), a further photopolymerizable or photocrosslinkable liquid is preferably used. The further photopolymerizable or photocrosslinkable liquid may be identical to or different from the liquid used in the first step. The latter either with regard to the photopolymerizable or photocrosslinkable substance, with regard to the concentration of individual components or with regard to the cell type or types used. The same also applies to photopolymerizable or photocrosslinkable liquids used in further steps.

As used herein, “photopolymerizable” should be understood to mean that the corresponding substance contains monomers which can be polymerized by the action of electromagnetic radiation and, if appropriate, the presence of a photoinitiator. Similarly, “photocrosslinkable” should be understood to mean that an oligomer or polymer can be crosslinked by the action of electromagnetic radiation and, if appropriate, the presence of a photoinitiator.

In one embodiment of the 3D scaffold according to the invention, it is preferred that the matrix of the biocompatible polymer is built up by the following steps (preferably in the order indicated):
(I) Introduction of a first photopolymerizable or photocrosslinkable liquid into a reaction vessel,
(II) Immersion or presence of a support plate on which the 3D scaffold is to be built up in the first photopolymerizable or photocrosslinkable liquid(s);
(III) Irradiation of electromagnetic radiation selectively, in a layer or holographically in the areas where curing of the liquid is desired
(IV) Creation of a polymerized or cross-linked structure by electromagnetic radiation,
(V) Introduction of a further photopolymerizable or photocrosslinkable liquid into a (further) reaction vessel,
(VI) Immersion or presence of the structure produced in step (IV) on the support plate in the further photopolymerizable or photocrosslinkable liquid,
(VII) Irradiating electromagnetic radiation selectively, in a layer or holographically in the areas in which curing of the further liquid is desired,
(VIII) Producing a further polymerized or crosslinked structure by the electromagnetic radiation in step (VII),
(IX) Repeating steps (V) to (VIII) with a further photopolymerizable or photocrosslinkable liquid in each case until the 3D scaffold is produced.

The polymerized or cross-linked structures produced in the repeated steps are preferably connected to each other by covalent bonds. However, non-covalent bonds, for example based on physical interactions, are also possible.

By repeating the process steps in which polymerization or crosslinking of the photopolymerizable or photocrosslinkable liquids takes place, a layered, punctual or holographic structure of the 3D scaffold is achieved. It is possible to create a 3D scaffold with a complex structure in which the various cell types used are located at different, desired positions within the 3D scaffold. Furthermore, undercuts and overhanging structures can also be formed, since polymerization or crosslinking of the photopolymerizable or photocrosslinkable liquid can take place in a certain layer or also certain point during punctual irradiation, even if no already polymerized or crosslinked material is arranged underneath, but only liquid that has not yet been polymerized or crosslinked. In this way, it is also possible according to the invention to build up a blood vessel structure network. There is no polymerization or crosslinking of a photopolymerizable or photocrosslinkable liquid present outside the layer or dot; instead, only the photopolymerizable or photocrosslinkable liquid located inside the layer or dot is polymerized or crosslinked.

The photopolymerizable or photocrosslinkable liquids used preferably each contain one or more biological cell types. If polymerization or crosslinking occurs as a result of exposure to electromagnetic radiation, the cells contained in the liquid are also embedded in a corresponding polymer. By using several photopolymerizable or photocrosslinkable liquids with preferably one or more different biological cell types, a complex 3D scaffold in the form of a biological breast implant can be constructed, which also has a blood vessel network. The use of a matrix of the biocompatible polymer enables the directional structure of the 3D scaffold according to the invention.

The matrix of the biocompatible polymer can be composed of a homogeneous material and thus only comprise a polymer of a single type, but can also be composed of a heterogeneous material of biopolymers of different types, whereby only the matrix-forming material without the cell types embedded in it is meant here.

A biocompatible polymer is understood to be a biological or biocompatible polymer. In this context, “biocompatible” is understood to mean that it does not affect the lifespan of the biological cells, in particular that it does not have a toxic effect on the biological cells. Furthermore, it is preferred that the biocompatible polymer is a biocompatible hydrogel. A hydrogel is understood to be a gel made of a water-insoluble polymer that can bind water. Furthermore, it is preferred that the biocompatible polymer is a biodegradable or digestible polymer. This allows the polymer to be gradually degraded or digested after implantation in the human body, so that only the autologous cells remain as the implant.

The photopolymerizable or photocrosslinkable substance in the photopolymerizable or photocrosslinkable liquid is preferably one which has a photoreactive group which can form covalent bonds with other photoreactive groups.

In one variant, the photoreactive group is an acrylic group by means of which the polymerization or crosslinking is effected. That is, the photopolymerizable or photocrosslinkable substance is preferably one of the following group: methacrylic acid, methacrylates, methyl acrylates, ethyl acrylates, hydroxyethyl acrylates, butyl acrylates, trimethylolpropane acrylates, triacrylic acrylates and polyacrylates (PA) in general.

The substance to be polymerized or crosslinked can be a polymer, an oligomer or a monomer. Preferably, these are carbon-based substances. In the case of monomers, photopolymerization is used. In the case of polymers or oligomers, photocrosslinking is preferred.

The following monomers, for example, can be used as monomers to be polymerized: Acrylamides, vinyl chloride, ethylene, propylene, isoprene, caprolactam, caprolactone lactide, all amino acids, (de)oxyribonucleotides, glucose, or all monosaccharides as well as the aforementioned acrylates, wherein caprolactone, lactide, monosaccharides, (de)oxyribonucleotides are preferred for reasons of biodegradability of the resulting polymers.

The following oligomers or polymers can be used: Polycaprolactone, polylactide, polyethylene glycol (PEG), polyethylene (PE), polypropylene (PP), polyketone (PK), polyvinyl chloride (PVC), polystyrene (PS), polytetrafluoroethylene (PTFE), polymethyl methacrylate (PMMA), polycarbonate (PC), polyethylene terephthalate (PET) and polyurethane (PU). Synthetic polymers such as silicones, polydimethylsiloxane (PDMS) or resins such as melamine or melamine-formaldehyde resins are also suitable as starting materials. Furthermore, biopolymers such as proteins, DNA, RNA, carbohydrates and carbohydrate derivatives, collagens, fibrins, alginates, gelatine, hyaluronic acids or polylactides are suitable as starting substances. Instead of the aforementioned polymers, the monomer precursors or oligomer precursors of these polymers can also be used as starting substances, provided that they can be provided in a stable manner in the solid or liquid aggregate state. By introducing a photoreactive group, e.g. an acrylic group, into the starting substance, it is made photopolymerizable or photocrosslinkable. The radiation-induced coupling of the acrylic residues between different molecules of the starting substance produces a polymerized or cross-linked matrix. Polycaprolactone, polylactides and all biological polymers can be used as polymers for reasons of biodegradability.

The starting substance supplemented by the photoreactive group is used in liquid form, whereby different viscosities are possible. This means that the process described here is not limited to photopolymerizable or photocrosslinkable liquids with a specific viscosity, but that low-viscosity liquids can also be used. Both Newtonian and non-Newtonian liquids can be used.

The liquids can be solutions or colloid-disperse mixtures, such as suspensions. The liquids can have an aqueous to oily character. This is determined, among other things, by the choice of starting substances and their particle size.

In order to achieve photopolymerization or photocrosslinking of the starting substance carrying a photoreactive group, a radical former (a so-called photoinitiator) is preferably used, which forms radicals at a selected wavelength of the electromagnetic radiation used in the process. Suitable radical formers are, for example, anthrone derivatives such as violanthrone or isoviolanthrone, fluorescein, rubrene, anthrazine derivatives, tetrazene derivatives, benzanthrone, benzanthronil, eosin, levolinic acid derivatives, phosphine derivatives, mono- and bis-acyl phosphines, in particular lithium phenyl-2,4,6-trimethylbenzoyl phosphinate, metallocenes, acetophenones, benzophenones, xanthones, quinones, ketone derivatives, hydroxyketones, aminoketones, benzoyl peroxides, pyridine salts, phenylglyoxylates and/or iodonium salts.

Preferably, in addition to the radical former, a vinyl macromer and an amine-based co-initiator are also used in order to allow the photopolymerization or photocrosslinking to take place in a particularly suitable manner. Suitable co-initiators include ascorbic acid and tertiary amine derivatives such as methyldiethanolamine or tetraethylamine.

In addition, a substance can be added to the photopolymerizable or photocrosslinkable liquid that prevents photopolymerization or photocrosslinking of deeper, liquid layers. In this way, the liquid solution remains liquid outside the focal plane, even if it is in the irradiation range of the focal plane above it. This works by absorbing the substance at the wavelength at which polymerization takes place (polymerizing wavelength). The interception takes place in the focal plane so that no penetration of the polymerizing wavelength into deeper layers is possible. All substances that absorb at the desired wavelength, such as dyes, are suitable.

Furthermore, in one variant, it is possible for the one photopolymerizable or photocrosslinkable liquid and/or one of the other photopolymerizable or photocrosslinkable liquids and/or another liquid, which does not have to be photopolymerizable, to have a temperature-sensitive gelling agent. In particular, the use of an inverse-temperature-sensitive (also referred to as reverse-temperature-sensitive) gelling agent is envisaged. Such a gelling agent becomes more solid with increasing temperature. By heating the reaction vessel, the reaction liquid solidifies and forms an initially only metastable gel. If the liquid is not photopolymerized or photocrosslinked at the same time, the metastable gel can be liquefied again and pumped out by subsequently cooling the 3D scaffold. With conventional temperature-sensitive gelling agents, the temperature conditions to be applied are exactly the opposite. For example, a support structure can be created if required so that suspended structures can be produced. If, on the other hand, the metastable gel is at least partially irradiated with electromagnetic radiation at a suitable wavelength, photopolymerization occurs so that the metastable gel is converted into a stable gel or polymer in these areas.

In other words, the temperature-sensitive, in particular inverse temperature-sensitive, gelling agent and temperature control of the reaction chamber make it even easier to work with hanging sections and undercuts or cavities. In this variant, it is still possible to work with liquid structures as a support.

It is also possible to provide a temperature gradient so that a metastable gel is not formed in all areas of the liquid mixed with the temperature-sensitive, in particular inverse temperature-sensitive, gelling agent. Even more complex structures can be produced with the aid of such a gradient.

The aforementioned individual components can be contained as individual substances in the photopolymerizable or photocrosslinkable liquid. Alternatively, it is also possible to realize the substances or groups preferably used for gel formation in a single polymer by appropriate synthesis. Instead of a mixture of individual components, such a polymer would then have different functional groups which combine all the functions required or preferably to be used for photopolymerization or photocrosslinking. Furthermore, it is also conceivable to provide only some of the functions or groups preferably used for photopolymerization or photocrosslinking in a polymer and to admix other functions or groups preferably to be used for photopolymerization or photocrosslinking in separate individual components of the photopolymerizable or photocrosslinkable liquid.

As an alternative or in addition to the formation of cavities through the use of a gelling agent, enzymes can also be used to digest the polymer. The principle is as follows: A 3D scaffold with cavities/undercuts (e.g. a channel system) is printed as a solid body by filling all cavities with a sacrificial material during printing, which can be dissolved later (i.e. after printing is complete) by administering the correct enzyme. The sacrificial material is, for example, a digestible polymer that is digested by adding a digesting enzyme. This is an elegant strategy for creating cavities using stereolithographic printing techniques. For example, a hyaluronidase (digesting enzyme) can digest hyaluronic acid (sacrificial material) to create a cavity at the point in the 3D scaffold where the hyaluronidase is inserted. This principle is already described in the patent application with the official file number DE 10 2019 200 792.9.

Alternatively, a photoblocker can also be used in the photopolymerizable or photocrosslinkable liquid to create a cavity/undercut, whereby the photoblocker restricts the curing depth of the photopolymerizable or photocrosslinkable liquid. In this way, curing can also be carried out by irradiation in one layer without curing all areas in the irradiation depth that lie in the beam path. The possibility of layer-by-layer curing increases the printing speed immensely compared to point-by-point processes.

In one variant, the further photopolymerizable or photocrosslinkable liquid is preferably introduced into a reaction vessel only after the photopolymerizable or photocrosslinkable liquid previously present in the reaction vessel (this can be, for example, the one photopolymerizable or photocrosslinkable liquid or a further photopolymerizable or photocrosslinkable liquid) has been removed from the reaction vessel. For this purpose, it is possible, for example, that a pump is provided which pumps an already used photopolymerizable or photocrosslinkable liquid out of the reaction vessel and pumps a new further photopolymerizable or photocrosslinkable liquid into the reaction vessel. Instead of a single pump, two or more different pumps can also be used for such processes. Alternatively, the entire 3D scaffold to be built up can also be built up on a carrier plate, which can be moved in such a way that the 3D scaffold is successively moved into different reaction vessels filled with photopolymerizable or photocrosslinkable liquid and the light-based, structuring curing is carried out there in each case, as already described above.

In one variant, an optical system is arranged between a source for the electromagnetic radiation (radiation source), which serves to generate the one and/or the other electromagnetic radiation, and the reaction vessel, which optical system serves to focus the electromagnetic radiation onto the respective focal plane in the reaction vessel. In one variant, it is envisaged that this optical system can be refocused in order to change the curing layer within the reaction vessel. Such refocusing can be achieved, for example, by changing the distance between the optical system and the radiation source. A computer-controlled stepper motor can be provided to impart a corresponding movement of the optical system. The optical system can, for example, be a system of optical lenses or—in a particularly simple design case—a single focusing lens. Alternatively, the curing point or layer can be static and the 3D scaffold to be built can be moved relative to the electromagnetic radiation.

In a further manufacturing variant of the 3D scaffold according to the invention, the one and/or the other electromagnetic radiation is directed to a defined and predeterminable area which lies within the one photopolymerizable or photocrosslinkable liquid and/or the other photopolymerizable or photocrosslinkable liquid. In other words, a specific radiation pattern can be predetermined which strikes the photopolymerizable or photocrosslinkable liquid and serves to polymerize or crosslink the liquid at these points to form the biocompatible polymer. Such a radiation pattern can be generated, for example, by using masks or apertures, but also by using pulsed radiation or digital modulation of a radiation signal. Polymerization or crosslinking occurs in the areas of the photopolymerizable or photocrosslinkable liquid that are hit by the radiation. In the other areas not affected by the radiation, however, the photopolymerizable or photocrosslinkable liquid remains in its non-polymerized or non-crosslinked state. As a result, the radiation defines the areas where the polymerized or crosslinked structure is printed. With this type of light-assisted printing, much higher resolutions are possible than is the case with the methods known from the prior art. The resolution depends on the wavelength of the radiation used. Even when large wavelengths are regularly used, it is better than the resolution that can be achieved with the conventional methods known from the state of the art. The more precisely the radiation source can be focused, the greater the resulting resolution. For example, very high resolutions can be achieved with a laser.

If required, the electromagnetic radiation can be directed to the respective layer or point via mirrors.

The selected radiation pattern can be provided by a computer program, for example. It is therefore conceivable that a user could create the 3D framework to be produced using a CAD program. The digital object created in this way is then cut into individual irradiation planes by a suitable computer program. Furthermore, a specific photopolymerizable or photocrosslinkable liquid is assigned to each plane or different locations of each plane. This information is used to create control information for a printer by means of which the described process is carried out. This control information specifies when which photopolymerizable or photocrosslinkable liquid must be introduced into the reaction vessel. Furthermore, this control information specifies when which image of an irradiation plane is to be projected onto the respective focal plane in the reaction vessel. In this way, the 3D scaffold previously created on the computer can be converted into a real 3D scaffold.

In one variant, more than one polymerized or crosslinked structure is produced in the same layer. For this purpose, a first photopolymerizable or photocrosslinkable liquid is polymerized or crosslinked first. The 3D scaffold to be built up is then introduced into a second photopolymerizable or photocrosslinkable liquid. The areas of the 3D scaffold to be built up that were not previously irradiated and therefore do not yet have a polymerized or cross-linked structure are now preferably irradiated. This allows different materials and cell types to be arranged in one and the same layer or in layers on top of each other, depending on how far the 3D scaffold to be built up is introduced into the second liquid. Consequently, several polymerized or cross-linked structures are formed in one and the same layer or 3D scaffold, which have different materials and cell types. In this way, it is also possible to incorporate complex structures, such as blood vessel networks, into the 3D scaffold. Subsequently, the 3D scaffold to be built up can be introduced into a further photopolymerizable or photocrosslinkable liquid with a further cell type, and so on. In this way, it is possible to place appropriate materials and cell types at any location within the 3D scaffold to enable the construction of a complex biological breast implant that closely resembles human body tissue.

In one variant, the first electromagnetic radiation and/or the further electromagnetic radiation have a wavelength in the range from 200 nm to 1000 nm (i.e. a wavelength lying between the UV range and the infrared range), more preferably in the range from 350 nm to 800 nm. With such wavelengths, the substances preferably used as radical formers can be excited particularly well, so that radicals are formed in order to enable polymerization or crosslinking of starting substances carrying acrylic residues.

Other suitable wavelengths of the electromagnetic radiation used are in the range from 250 nm to 950 nm, in particular from 250 nm to 850 nm, in particular from 300 nm to 800 nm, in particular from 300 nm to 750 nm, in particular from 300 nm to 700 nm, in particular from 350 nm to 650 nm and, in particular, from 350 nm to 400 nm.

The radiation used for polymerization or crosslinking can comprise the same wavelength or different wavelengths from the aforementioned wavelength range in order to enable suitable polymerization of the different photopolymerizable or photocrosslinkable liquids. The individual radiations can be generated by different radiation sources or by one and the same radiation source. It is also possible to use different wavelengths successively within a layer (and thus within a focal plane) in order to polymerize or crosslink different photopolymerizable or photocrosslinkable liquids in the same layer if a heterogeneous layer is to be formed from different polymerized or crosslinked structures.

As can be seen from the previous illustration of the manufacture of the 3D scaffold according to the invention, the step of 3D printing the 3D scaffold can be carried out fully automatically, so that user intervention is not required. This further facilitates the application of the method.

The period of time during which the electromagnetic radiation is irradiated onto the respective focal plane can be adapted to the respective requirements of the photopolymerizable or photocrosslinkable liquids used. This means that each material is allowed the time required for the desired polymerization or crosslinking for curing.

If the 3D scaffold is created on a carrier plate, this carrier plate can be completely lifted out of the remaining liquid in the reaction vessel at the end of the manufacturing process. The user can then remove the 3D scaffold from the carrier plate.

In one embodiment of the 3D adipose tissue scaffold according to the invention, it is preferred that human breast tissue has been partially or completely removed (mastectomy) for prevention or as a result of a disease. It is particularly preferred that the disease is breast cancer. The removal of the breast tissue for the prevention of a disease preferably results from the determination of a genetic disposition which, for example, increases the risk of cancer, preferably a genetic disposition for breast cancer. In a further embodiment of the 3D adipose tissue scaffold according to the invention, it is preferred that the cells of the plurality of cell types are autologous cells. Autologous cells are understood to be the body's own cells, i.e. the cells used to construct the 3D adipose tissue scaffold are cells taken from the patient who is to receive the breast implant. In this way, rejection reactions of the implanted breast implant are unlikely.

The cells of the plurality of cell types are preferably selected from the group consisting of adipocytes, adipocyte progenitor cells, adipocyte stem cells, fibroblasts, endothelial cells and a combination thereof. It is preferred that adipocyte stem cells are used for light-based, structuring curing, which then develop into adipocytes. In order to support the further development of the adipocytes into adipocyte stem cells, cellular messengers can be used according to the invention. Preferably, so-called adipogenic differentiation factors are used for this purpose. These comprise signaling molecules from the classes of growth factors and hormones. The adipogenic factors are preferably added to the nutrient fluid for the culture of the 3D adipose tissue scaffold in which the adipocyte stem cells are located.

The cell types mentioned are preferably introduced into one or more photopolymerizable or photocrosslinkable liquids. Each cell type can be introduced into one photopolymerizable or photocrosslinkable liquid at a time. Alternatively, two (or more) cell types can also be introduced into one photopolymerizable or photocrosslinkable liquid.

In one embodiment of the 3D adipose tissue scaffold according to the invention, it is preferred that a first photopolymerizable or photocrosslinkable liquid containing adipocyte stem cells and a second photopolymerizable or photocrosslinkable liquid containing endothelial cells or fibroblasts are used for the light-based, structuring curing. It is further preferred that the second photopolymerizable or photocrosslinkable liquid contains endothelial cells. In the latter case, fibroblasts can either be additionally contained in the first photopolymerizable or photocrosslinkable liquid, or a third photopolymerizable or photocrosslinkable liquid with fibroblasts is additionally used for the light-based, structuring curing.

Preferably, however, the adipocyte stem cells and the fibroblasts are placed in a single photopolymerizable or photocrosslinkable liquid and the endothelial cells are placed in a different photopolymerizable or photocrosslinkable liquid. In this way, the cells that perform the tissue function (adipocytes) can be placed at different locations in the 3D scaffold from the cells for blood vessel formation during the construction of the 3D scaffold. This makes it possible to create a vascular network.

The 3D adipose tissue scaffold according to the invention preferably comprises adipocytes and/or adipocyte stem/progenitor cells, and a further cell type. The further cell type is preferably selected from fibroblasts and endothelial cells, with endothelial cells being preferred. More preferably, the 3D adipose tissue scaffold according to the invention comprises adipocytes and/or adipocyte stem/progenitor cells, fibroblasts and endothelial cells. The adipocytes are responsible for fat/energy storage and the hormonal activity of the 3D scaffold. The fibroblasts in the 3D scaffold are responsible for the secretion of extracellular matrix and for supporting vascularization. The endothelial cells are responsible for blood vessel formation (vasculogenesis and angiogenesis) and tissue supply.

Furthermore, in the 3D adipose tissue scaffold according to the invention, it is preferred that the light-based, structuring curing is a stereolithographic 3D printing process. As mentioned above, the stereolithographic 3D printing process is preferably a projection-based exposure process.

An exposure process using structuring curing of a photopolymerizable or photocrosslinkable substance to form the biocompatible polymer—as used according to the invention—has the advantage over other biological printing processes, such as the extrusion process, the inkjet process and the laser-assisted forward transfer process, of an outstanding printing speed, i.e. how much volume of the object to be printed can be built up per unit of time. Unique to light-based structuring curing is the combination of high printing speed and high spatial resolution. The efficiency of the process is based on optical laws, and the use of commercially available optics enables superior resolution down to the sub-micrometer range.

In one embodiment of the breast implant according to the invention, it is preferred that abnormal breast tissue is removed from the patient in one step. Furthermore, it is preferred that healthy adipocyte stem cells, fibroblasts and endothelial cells are removed from the patient. The removed healthy cells are then preferably cultured. By adding the cultured cells, the various photopolymerizable or photocrosslinkable liquids are then preferably produced, as described above, which are used to construct the 3D scaffold according to the invention. After building the 3D scaffold, the interior of the formed blood vessel network is preferably enzymatically digested to expose the channels. In one variant, the 3D scaffold is cultivated in a further intermediate step to form tissue structures. The implant can then be implanted in the patient.

The present invention relates not only to the 3D scaffold according to the invention, but also to the use of a 3D adipose tissue scaffold as a breast implant in humans, wherein the 3D adipose tissue scaffold comprises a matrix of a biocompatible polymer which is colonized with a plurality of cell types, the colonization with the plurality of cell types taking place during the construction of the matrix of biocompatible polymer with the formation of a blood vessel structure network within the 3D adipose tissue scaffold, characterized in that light-based, structuring curing of a photopolymerizable or photocrosslinkable liquid is used for the construction of the matrix of biocompatible polymer. This is preferably a non-medical indication. According to the invention, all the definitions and process steps for its manufacture mentioned above in connection with the 3D scaffold according to the invention should also apply to the 3D adipose tissue scaffold used in the application according to the invention.

### BRIEF DESCRIPTION OF THE DRAWINGS

Various results are shown in the figures, as described in more detail below in the example section:

FIGS. 1a to 1c show the differentiation of hASCs into adipocytes (control group in 2D culture).

FIGS. 2a and 2b show the histological and electron microscopic evidence of formed fatty tissue.

FIGS. 3a to 3c show the vascularization of the bioprinted adipose tissue.

### EXAMPLES

Production of a photopolymerizable or photocrosslinkable liquid (without cells):

Porcine gelatine type A (300 Bloom, Sigma-Aldrich, USA) is methacrylated as described in [1] and [2]. In brief: 10% w/v gelatine is dissolved in phosphate buffered saline (PBS) and heated to 50° C. Methacrylic anhydride (Sigma-Aldrich, USA) is added dropwise (0.1 mL/gelatin) and the reaction mixture is stirred for three hours to obtain methacrylated gelatin (GelMA). After the pH of the solution has been neutralized, it is dialyzed against distilled water (12-14 kDa cut-off membrane) for four days to desalinate and remove free methacrylate. It is then freeze-dried (Alpha 1-4 LDplus, Martin Christ, Germany, −60° C., 1 mbar) to obtain a long-lasting lyophilizate. Hyaluronic acid (HA) from Streptococcus Equi (molecular weight>1 MDa, Alfa Aesar, USA) is autoclaved to reduce the chain length and then methacrylated hyaluronic acid (HAMA) is synthesized according to a modified protocol by Poldervaart et al [3]. In brief, hyaluronic acid is dissolved in ultrapure water and the solution is adjusted to pH 9.0. Then 2.0 ml/gHA methacrylic anhydride dissolved in dimethyl sulfoxide (VWR, United Kingdom) is added and stirred for 24 hours at room temperature. The product is purified by dialysis against ultrapure water and then lyophilized. The photoinitiator lithium phenyl 2,4,6-trimethylbenzoyl phosphinate (LAP) is synthesized as described elsewhere [4][5]. The degree of functionalization of the synthesized GelMA and HAMA is verified by 1H-NMR using a Bruker Avance III at 500 MHz (Bruker Corporation, USA).
[1] a I. Van Den Bulcke, B. Bogdanov, N. De Rooze, E. H. Schacht, M. Cornelissen, and H. Berghmans, “Structural and rheological properties of methacrylamide modified gelatin hydrogels,” Biomacromolecules, vol. 1, no. 1, pp. 31-38, 2000, doi: 10.1021/bm990017d.
[2] H. Shirahama, B. H. Lee, L. P. Tan, and N. J. Cho, “Precise tuning of facile one-pot gelatin methacryloyl (GelMA) synthesis,” Scientific Reports, vol. 6, no. August, pp. 1-11, 2016, doi: 10.1038/srep31036.
[3] M. T. Poldervaart et al, “3D bioprinting of methacrylated hyaluronic acid (MeHA) hydrogel with intrinsic osteogenicity,” PLoS ONE, vol. 12, no. 6, pp. 1-15, 2017, doi: 10.1371/journal.pone.0177628.
[4] T. Majima, W. Schnabel, and W. Weber, “Phenyl-2,4,6-trimethylbenzoylphosphinates as water-soluble photoinitiators. Generation and reactivity of O□P (C6H5) (O—) radical anions,” Die Makromolekulare Chemie, vol. 192, no. 10, pp. 2307-2315 October 1991, doi: 10.1002/macp.1991.021921010.
[5] B. D. Fairbanks, M. P. Schwartz, C. N. Bowman, and K. S. Anseth, “Photoinitiated polymerization of PEG-diacrylate with lithium phenyl-2,4,6-trimethylbenzoylphosphinate: polymerization rate and cytocompatibility,” Biomaterials, vol. 30, no. 35, pp. 6702-6707, 2009, doi: 10.1016/j.biomaterials.2009.08.055.

### Production of Cell Cultures:

Various cell cultures are produced. For this purpose, human adipose stem cells (hASCs), dermal fibroblasts (hDFs) and umbilical vein endothelial cells (HUVECs) are purchased from Lonza (Switzerland) and expanded under standard cell culture conditions (37° C. and 5% CO2). The medium is completely replaced every two to three days. All cells are passaged at 90% confluence and used for bioprinting at a maximum passage number of 4. hASCs and HUVECs are thawed and expanded in Endothelial Cell Growth Medium 2 (ECGM 2—PromoCell, Germany) supplemented with 100 IU/mL penicillin and 100 μg/mL streptomycin (Gibco, USA). hDFs are thawed and expanded in Dulbecco's Modified Eagle's Medium with 4.5 g/mL glucose (DMEM high glucose—Corning, USA) supplemented with 10% v/v fetal calf serum (Corning, USA) and 100 IE/mL penicillin and 100 μg/mL streptomycin (Gibco, USA).

Preparation of the Photopolymerizable or Photocrosslinkable Liquids with the Prepared Cell Cultures:

A total of two different photopolymerizable or photocrosslinkable liquids, each mixed with the cell cultures cultivated in the previous step, are produced. The first liquid should contain the hASCs and the hDFs, the second liquid the HUVECs. These liquids are prepared by dissolving and then diluting the respective lyophilized material in PBS. The cells are then mixed directly into the photopolymerizable or photocrosslinkable liquids shortly before the printing process. The compositions of the resulting liquids are listed in Table 1.

TABLE 1
Composition of the photopolymerizable or photocrosslinkable liquids.
The components and concentrations in % by weight are given for the
various liquids used to manufacture the individual compartments of
the breast implant. Cell types and concentrations for cell-loaded
photopolymerizable or photocrosslinkable liquids are indicated.

| Compartment | Composition | Cells |
|---|---|---|
| Adipose tissue | 5% GelMA + 1% HAMA + | 9.9 * 10⁶/mL hASCs + |
| | 0.1% LAP | 0.1 * 10⁶/mL hDFs |
| Blood vessels | 1.5% HAMA + 0.05% | 20 * 10⁶/mL |
| | LAP | HUVECs |
| Binding gel | 5% GelMA + 0.1% LAP | — |

LAP: lithium phenyl 2,4,6-trimethylbenzoyl phosphinate

### 3D Bioprinting of the 3D Adipose Tissue Scaffold as a Breast Implant

Three-dimensional 3D adipose tissue scaffolds are designed using Rhinoceros 6 (Robert McNeel & Associates, Seattle, USA). The STL file is exported and the projection masks are created by the bioprinter software. Bioprinting is carried out using a bioprinting platform from Cellbricks GmbH, as described in EP 3 018 531 A1. The printed 3D adipose tissue scaffolds are embedded in a soft hydrogel by overlaying them with 400 μL of the embedding gel in 24-well plates and post-curing for 10 minutes with a 385 nm light source (2.5 mW/cm2). 500 μL of ECGM2 containing 75 U/mL hyaluronidase (STEMCELL Technologies, Canada) is added to the wells of the plate and incubated overnight under cell culture conditions to allow enzymatic digestion of the interior of the fabricated channel structures, resulting in the release of the embedded HUVECs and their subsequent adhesion to the channel walls.

### In Situ Differentiation of the 3D Adipose Tissue Scaffold

After printing, fabricated 3D adipose tissue scaffolds are cultivated in specialized adipogenic culture media to form adipose tissue in situ. The first step is a 3-day acclimatization in ECGM2. Subsequently, the prints are cultivated in adipogenic induction medium based on ECGM2 with the addition of 0.25 mM isobutyl-1-methylxanthine (IBMX), 0.1 μM insulin, 1 μM rosiglitazone, 0.2 nM triiodothyronine and 1 μM dexamethasone. This is followed by a complete medium change to adipogenic differentiation medium. This has the same composition as the induction medium, but does not contain IBMX. The printed 3D adipose tissue scaffolds are cultivated in adipogenic differentiation medium for the entire remaining culture period (up to 4 weeks after printing) with daily complete medium changes.

### Bioanalytical Methods and Results:

### a) the 3D Bioprinted Breast Implant Model is Vital Over the Entire Culture Period of 4 Weeks and Shows High Cellular Dynamics:

Live/dead staining (calcein-AM, Hoechst33342 & propidium iodide) of the bio-printed adipose tissue models and fluorescence microscopy shows a high proportion of living cells and a low content of dead cells 3, 13 and 27 days after printing. In addition to the qualitative detection, living and dead cell populations are quantified and result in >90% of living cells in the bio-printed adipose tissue models at all analysis times. Vitality can be indirectly detected via high cellular activity in the printed constructs. Non-invasive automated microscopy shows dynamic cell behavior with migrating and interacting cells (clusters), and the formation of multicellular tissue-like structures. In particular, outgrowth of cells from the bioprinted constructs into the surrounding cell-free embedding hydrogel is observed. Embedding the bio-printed adipose tissue models in a hydrogel, which simulates the ECM (collagenoids and hyaluronic acid as material base) and biomechanics (similar substrate stiffness) of adipose tissue, offers the opportunity to use an in vitro setup, which is relevant for later implantation in native adipose tissue.

Experimental procedure: The differentiation medium is used as a base for staining. Calcein-AM and Hoechst33342 are diluted 1:5000 (0.1 μl per 500 μl), while PI is used at a dilution of 1:50 (10 μl per 500 μl). The staining is carried out in a 24-well plate. 500 μl of this color solution is added to the bioprinted adipose tissue model. The incubation takes place at room temperature and lasts three hours, with the color solution being resuspended every hour. The live-dead staining is photographed using an automated fluorescence microscope (NYONE®, SynenTec) and objectified by the YT-Software® with regard to the staining intensity. This is used to quantify the live and dead cell populations.

b) after Sufficient Maturation, the 3D Bioprinted Adipose Tissue Contains Adipocytes and is Vascularized:

Adipose tissue precursors created by 3D bioprinting can be successfully matured into mature adipose tissue characterized by the presence of adipocytes using an adapted differentiation protocol. Imprinted adipose stem cells (hASCs) are differentiated into adipocytes by culture in an adipogenic medium, detectable by the appearance of cellular fat vesicles, the extent of which increases as the culture progresses. This process can be detected in control cultures in the Petri dish as well as in the bioprinted adipose tissue by light microscopy and by fat-specific Nile Red staining. After several weeks of culture, the imprinted endothelial cells (HUVECs) can not only be detected in the specified channel system structured by 3D printing, but also develop capillary-like networks that permeate other parts of the bio-printed adipose tissue. This is detected by live cell staining and specifically by anti-CD31 staining in the bioprinted adipose tissue, even after several weeks of culture. Adipose and vascular tissue structures can also be revealed by scanning electron microscopy and histology. In particular, stained thin sections of mature bioprinted adipose tissue with a loose, fat vacuole-rich tissue structure can be seen not only within the constructs, but also surrounding them in the embedding hydrogel. Immunohistochemical detection of mature adipocytes (anti-S100 staining), endothelial cells (anti-CD31 staining) and the formation of new cellular extracellular matrix (Masson-Goldner trichrome staining) confirms the maturation process of the bioprinted constructs to vascularized adipose tissue.

### Experimental Implementation:

Nile Red staining: Nile Red fat staining is used to objectify the amount of fat vesicles and their visualization. Nile Red is a fluorescent phenoxazine dye (excitation: 515-560 nm; emission: >590 nm), which can stain fat vesicles due to its lipophilicity. An adipose tissue print is transferred to a 24-well plate and incubated for 30 min at room temperature in 10% formalin solution for fixation. The print is then washed with 500 μl PBS for 10 min, after which it is permeabilized for a further 10 min in 500 μl 0.1% Triton X-100 solution. After a further PBS washing step, the print is incubated for 30 min at room temperature in the Nile Red staining solution (0.25 mg/ml in pure ethanol). A final wash step with PBS is followed by a 10 min incubation at room temperature with DAPI (4′,6-diamidine-2-phenylindole; 10 μg/ml in PBS) to counterstain the nuclei.

The Nile Red fat staining is also photographed using the automated fluorescence microscope NYONE® (SynenTec, excitation: 562 nm (lime), emission: 628 nm (red)) and objectified by the YT-Software® with regard to staining intensity and extent.

Sample preparation for (immuno) histochemical staining: An adipose tissue print is transferred to a 24-well plate and first incubated for 30 min at room temperature in 10% formalin solution for fixation. The print is then washed with 500 μl PBS for 10 min before being transferred to a sample cassette for dehydration via an ascending alcoholic series (50% ethanol, 70% ethanol, 96% ethanol, xylene, 5 min each). The dehydrated print is embedded in kerosene and the resulting block is cut by microtome. The resulting thin sections are mounted on glass slides, air-dried and stored in a cool, dark place until staining.

Immunohistochemical staining: Dehydrated thin sections are rehydrated using a descending alcoholic series, a subsequent wash step with ultrapure water and final incubation in PBS. Then permeabilize with a PBS+0.25% Triton X-100 wash step (10 min) and overlay the samples with 10% goat serum in PBS (30 min at room temperature) to block non-specific antibody binding. Staining solutions with one of the two primary antibodies used (mouse) against human CD31 or S100 are prepared in PBS+10% goat serum and applied to the thin sections overnight at 4° C. The samples are then completely exchanged for human CD31 or S100. This is followed by a complete exchange with the secondary antibody solution (goat anti-mouse, conjugated with horseradish peroxidase) and a further incubation step at room temperature for 1 h. The staining is completed by incubation with a chromogenic substrate solution (3,3′-diaminobenzidine) at room temperature for 10 min and subsequent rinsing with tap water for 5 min. The immunohistochemical staining is supplemented with a hematoxylin counterstain. For this purpose, the thin sections are incubated with a hematoxylin solution according to Mayer for 8 min at room temperature, then rinsed for 10 min with tap water and again briefly with ultrapure water. Finally, the stained thin sections are dehydrated with an ascending alcoholic series and capped with a non-aqueous medium.

Masson-Goldner trichrome staining: The dehydrated thin sections are rehydrated using a descending alcoholic series and a subsequent washing step with ultrapure water. They are then incubated with a hematoxylin solution according to Weigert for 15 min at room temperature and then rinsed for 8 min with tap water and again briefly with ultrapure water. In the next step, the thin sections are stained with an acid fuchsin-ponceau 2R-azophloxin solution for 4 min before being immersed in 1% acetic acid for a short color differentiation step. This is followed by staining with phosphomolybdic acid-orange G solution for 30 min with successive short differentiation steps in 1% acetic acid. The last staining step comprises incubation with light green solution and subsequent differentiation in 1% acetic acid. Wash for 1 min with running tap water and finally dehydrate the stained thin sections with an ascending alcoholic series. Finally, the sections are mounted with a non-aqueous mounting medium.

### DETAILED DESCRIPTION OF THE PREFERRED EMBODIMENTS

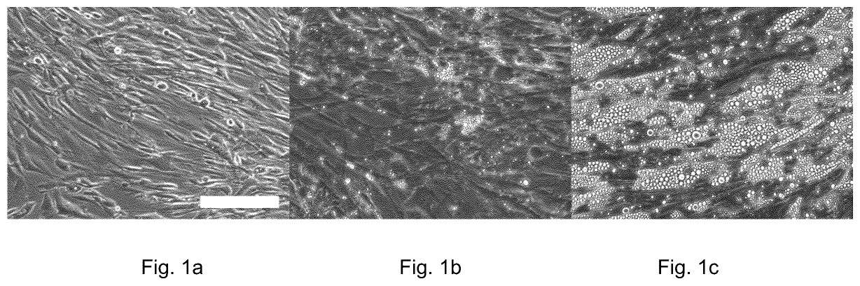

FIGS. 1a to 1c show the differentiation of hASCs into adipocytes (control group in 2D culture). A successful generation of adipocytes is recognizable by strong cell morphological changes, in particular the formation of intracellular fat vesicles, which manifest themselves microscopically as oil vesicles. As the culture time progresses under adipogenic conditions, there is increased fat vesicle formation and the development of larger vesicles, which is a characteristic of mature adipocytes. Images taken at different times in culture: 3 days (left), 6 days (center) and 10 days (right) after the start of differentiation. Scale bar: 100 μm.

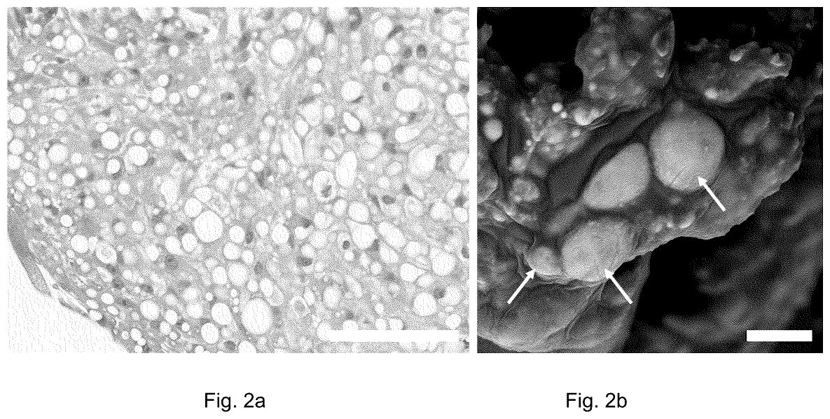

FIGS. 2a and 2b show the histologic and electron microscopic evidence of formed adipose tissue. Contrast staining with hematoxylin & eosin (left) of bioprinted adipose tissue after 4 weeks in culture shows a loose, fat vacuole-rich tissue with intermediate connective tissue components. Scale bar: 100 μm. Examination of the ultrastructure of the bioprinted adipose tissue by scanning electron microscopy (right) shows cellular components and in particular prominent fat vesicles (light gray, arrows) embedded in the print matrix (hydrogel, dark gray). Scale bar: 10 μm.

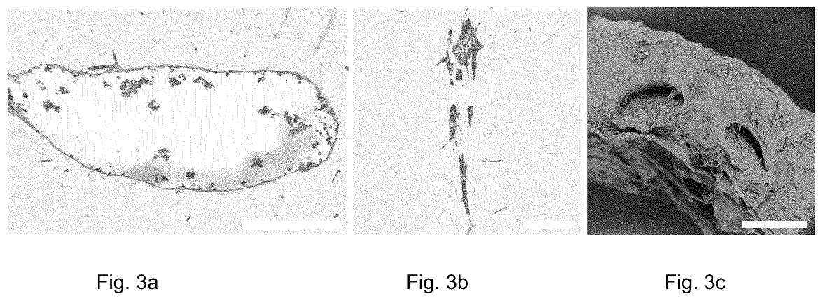

FIGS. 3a to 3c show the vascularization of the bioprinted adipose tissue. Endothelial cells used in bioprinting line the printed channels and thus form larger vessel-like structures, but also arrange themselves into microcapillary structures over the culture period. Immunohistological detection of structures formed by endothelial cells by anti-CD31 staining in the printed channel (left, cross-section, channel wall lined with cells marked with arrows), as well as in formed microcapillary cell clusters (center) in bio-printed adipose tissue after 4 weeks of culture. Scale bar: 200 μm. Scanning electron micrograph of a transverse section of the bio-printed adipose tissue model (right) reveals cross-sections of printed channels (marked with white arrows). Scale bar: 500 μm.

1. 3D adipose tissue scaffold for use as a breast implant for surgical, therapeutic, prophylactic or aesthetic restoration, reconstruction or replacement of human breast tissue, wherein the 3D adipose tissue scaffold comprises a matrix of a biocompatible polymer which is colonized with a plurality of biological cell types, the colonization with the plurality of cell types taking place during the construction of the matrix of biocompatible polymer with the formation of a blood vessel structure network within the 3D adipose tissue scaffold, characterized in that light-based, structuring curing of one or more photopolymerizable or photocrosslinkable liquids is used for the construction of the matrix of biocompatible polymer.

2. The 3D adipose tissue scaffold according to claim 1, wherein the human breast tissue has been removed for prevention or as a result of a disease.

3. The 3D adipose tissue scaffold according to claim 2, wherein the disease is breast cancer.

4. The 3D adipose tissue scaffold according to claim 1, wherein the cells of the plurality of cell types are autologous cells.

5. The 3D adipose tissue scaffold according to claim 1, wherein the cells of the plurality of cell types are selected from the group consisting of adipocytes, adipocyte stem/progenitor cells, fibroblasts, endothelial cells, and a combination thereof.

6. The 3D adipose tissue scaffold according to claim 5, wherein the cells of the plurality of cell types are adipocytes and/or adipocyte stem/progenitor cells, and fibroblasts and/or endothelial cells.

7. The 3D adipose tissue scaffold according to claim 1, wherein the light-based structuring curing is a stereolithographic 3D printing process.

8. The 3D adipose tissue scaffold of claim 7, wherein the stereolithographic 3D printing process is a projection-based exposure process.

9. The 3D adipose tissue scaffold according to claim 1, wherein a first photopolymerizable or photocrosslinkable liquid comprising adipocyte stem cells and a second photopolymerizable or photocrosslinkable liquid comprising endothelial cells or fibroblasts are used for the light-based, structuring curing.

10. The 3D adipose tissue scaffold according to claim 9, wherein the second photopolymerizable or photocrosslinkable liquid comprises endothelial cells.

11. The 3D adipose tissue scaffold according to claim 10, wherein a third photopolymerizable or photocrosslinkable liquid with fibroblasts is additionally used for the light-based, structuring curing.

12. The 3D adipose tissue scaffold according to claim 10, wherein the first photopolymerizable or photocrosslinkable liquid additionally contains fibroblasts.

13. The 3D adipose tissue scaffold according to claim 9, wherein further one or more messengers are used to differentiate the adipocyte stem cells into adipocyte progenitor cells and adipocytes.

14. The 3D adipose tissue scaffold according to claim 1, wherein a photoblocker is used in one or more of the one or more photopolymerizable or photocrosslinkable liquids.

15. Use of a 3D adipose tissue scaffold as a breast implant in humans, wherein the 3D adipose tissue scaffold comprises a matrix of a biocompatible polymer which is colonized with a plurality of cell types, the colonization with the plurality of cell types taking place during the construction of the matrix of biocompatible polymer with the formation of a blood vessel structure network within the 3D adipose tissue scaffold, characterized in that light-based, structuring curing of a photopolymerizable or photocrosslinkable liquid is used for the construction of the matrix of biocompatible polymer.
